(19) United States
(12) Patent Application Publication
(10) Pub. No.: US 2012/0003286 A1
(43) Pub. Date: Jan. 5, 2012

(54) COSMETIC CANDLE

(76) Inventor: Erez ZABARI, Rehovot (IL)

(21) Appl. No.: 13/153,560

(22) Filed: Jun. 6, 2011

Related U.S. Application Data
(62) Division of application No. 11/821,057, filed on Jun. 21, 2007, now abandoned.
Application No. 11/821,057 is a continuation-in-part of application No. PCT/IL2006/001088, filed on Sep. 21, 2006.

(30) Foreign Application Priority Data
Sep. 18, 2005 (IL) 170919

(51) Int. Cl. A61K 8/92; A61Q 19/00

(52) U.S. Cl. 424/401; 424/69

(57) ABSTRACT

A cosmetic candle comprising tiny grains embedded therein. The grains impart a mechanical property that can be exploited for scrubbing skin to peel off old or dead tissue with a concomitant massaging of molten waxes into the skin. Crushed pits and seeds of olives, apricots, peaches, citrus fruits and/or plums are natural products that can be used as grains in embodiments of the invention. In addition, solid salt grains, such as natural salts from the Dead Sea, or sugar grains, may also be used to that effect.

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND

The present invention relates in general to fragranced candles and in particular to candles for cosmetic use.

Fragranced candles made of waxes and/or oils classified as cosmetic are common in the market place. Small amounts of warmed oil and/or wax can be directly massaged into the skin, leaving the skin smooth, soft and pleasantly scented. Therefore such candles not only appeal to the visual, tactile and olfactory senses but also can be used to treat the, skin.

### SUMMARY

The present invention comprises a cosmetic candle containing tiny grains embedded therein. These grains impart a mechanical property that can be exploited for scrubbing the skin of a user to peel off old or dead tissue, with a concomitant massaging of molten waxes into the skin. Crushed pits and seeds of olives, apricots, peaches, citrus fruits and/or plums are natural products that can be used as grains in embodiments of the invention. Solid salt grains, such as natural salts from the Dead Sea, or sugar grains, may also be used. Grains of plastic resins such as polyethylene although suitable for the scrubbing activity, are potentially emitters of soot and unpleasant odors and therefore should not be included in the ingredients of such a candle.

### DESCRIPTION

The cosmetic candle of the present invention contains tiny grains embedded therein. These grains impart a mechanical property that can be exploited for scrubbing the skin of a user to peel off old or dead tissue, with a concomitant massaging of molten waxes into the skin. Crushed pits and seeds of olives, apricots, peaches, citrus fruits and/or plums are natural products that can be used as grains in embodiments of the invention. Solid salt grains, such as natural salts from the Dead Sea, or sugar grains, may also be used to that effect.

Grains of plastic resins such as polyethylene although suitable for the scrubbing activity, are potentially emitters of soot and unpleasant odors and therefore should not be included in the ingredients of such a candle.

According to the invention, suitable ingredients for making the matrix of the cosmetic candle are any waxes or paraffin waxes as known in the art of candle making. Additionally, any of the following are materials that can be employed in the making of the candles: palm kernel wax, beeswax, and propolis wax. Other ingredients possibly included, impart specific cleansing, aromatic, soothing and other cosmetic characteristics to the candles. Among these are chamomile extract, propylene glycol, aloe vera gel, papaya extract, mango extract, melon extract, vanilla planifolia extract, soybean flour, soy oil, carnauba wax, fragranced olive oil, jojoba oil, evening primrose oil, propylparaben, shea butter, honey, tocopheryi acetate, sweet almond oil, Dead Sea salts, citrus extracts such as extracts of orange, lemon, tangerine, lime and or grapefruit, and any combination thereof.

In accordance with a preferred embodiment of the present invention pits of olives and apricots are coarsely crushed and mixed with salts from the Dead Sea. This mixture is further crushed to prepare a fine powder consisting of tiny grains. The powder is further mixed with a selection of molten waxes and oils from the list mentioned above and heated to a specified temperature, such that a homogeneous mixture is formed. The candle is further formed as is in the prior art and is preferably disposed in a container made of a light metal, ceramic or glass. A plain wick, typically a hemp wick or any suitable wick without a metal core is affixed to the bottom of the container. The container provides for an aesthetic appeal element, while affording additional safety and convenience in handling the candle while lit.

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND

The present invention relates in general to fragranced candles and in particular to candles for cosmetic use.

Fragranced candles made of waxes and/or oils classified as cosmetic are common in the market place. Small amounts of warmed oil and/or wax can be directly massaged into the skin, leaving the skin smooth, soft and pleasantly scented. Therefore such candles not only appeal to the visual, tactile and olfactory senses but also can be used to treat the skin.

### SUMMARY

The present invention comprises a cosmetic candle containing tiny grains embedded therein. These grains impart a mechanical property that can be exploited for scrubbing the skin of a user to peel off old or dead tissue, with a concomitant massaging of molten waxes into the skin. Crushed pits and seeds of olives, apricots, peaches, citrus fruits and/or plums are natural products that can be used as grains in embodiments of the invention. Solid salt grains, such as natural salts from the Dead Sea, or sugar grains, may also be used to that effect. Grains of plastic resins such as polyethylene although suitable for the scrubbing activity, are potentially emitters of soot and unpleasant odors and therefore should not be included in the ingredients of such a candle.

The cosmetic candle of the present invention contains tiny grains embedded therein. These grains impart a mechanical property that can be exploited for scrubbing the skin of a user to peel off old or dead tissue, with a concomitant massaging of molten waxes into the skin. Crushed pits and seeds of olives, apricots, peaches, citrus fruits and/or plums are natural products that can be used as grains in embodiments of the invention. Solid salt grains, such as natural salts from the Dead Sea, or sugar grains, may also be used to that effect.

Grains of plastic resins such as polyethylene although suitable for the scrubbing activity, are potentially emitters of soot and unpleasant odors and therefore should not be included in the ingredients of such a candle.

According to the invention, suitable ingredients for making the matrix of the cosmetic candle are any waxes or paraffin waxes as known in the art of candle making. Additionally, any of the following are materials that can be employed in the making of the candles: palm kernel wax, beeswax, and propolis wax. Other ingredients possibly included, impart specific cleansing, aromatic, soothing and other cosmetic characteristics to the candles. Among these are chamomile extract, propylene glycol, aloe vera gel, papaya extract; mango extract, melon extract, vanilla planifolia extract, soybean flour, soy oil, carnabua wax, fragranced olive oil, jojoba oil, evening primrose oil, propylparaben, shea butter, honey, tocopheryl acetate, sweet almond oil, Dead Sea salts, citrus extracts such as extracts of orange, lemon, tangerine, lime and or grapefruit, and any combination thereof.

In accordance with a preferred embodiment of the present invention pits of olives and apricots are coarsely crushed and mixed with salts from the Dead Sea. This mixture is further crushed to prepare a fine powder consisting of tiny grains. The powder is further mixed with a selection of molten waxes and oils from the list mentioned above and heated to a specified temperature, such that a homogeneous mixture is formed. The candle is further formed as is in the prior art and is preferably disposed in a container made of a light metal, ceramic or glass. A plain wick, typically a hemp wick or any suitable wick without a metal core is affixed to the bottom of the container. The container provides for an aesthetic appeal element, while affording additional safety and convenience in handling the candle while lit.

What is claimed is:

1. A method for treating the skin, comprising scrubbing the skin of a user with a cosmetic candle to peel off old or dead skin tissue with concomitant massaging of molten waxes from the cosmetic candle into the skin, wherein the cosmetic candle comprises:
a. a matrix comprising wax and at least one material that imparts cosmetic characteristics to the candle; and
b. a fine powder homogeneously embedded in the matrix.

2. The method of claim 1, wherein the cosmetic candle is installed in a container, wherein a wick is affixed to a bottom of the container.

3. The method of claim 1, wherein the material that imparts cosmetic characteristics comprises at least one cosmetic ingredient selected from the group of cosmetic ingredients consisting of propylene glycol, chamomile extract, Aloe vera gel, papaya extract, mango extract, melon extract, vanilla planifolia extract, soybean flour, soy oil, fragranced olive oil, jojoba oil, evening primrose oil, propylparaben, Shea butter, honey, tocopheryl acetate, sweet almond oil, citrus extract, and salts.

4. The method of claim 3, wherein the material that imparts cosmetic characteristics comprises a citrus extract selected from the group consisting of orange extract, lemon extract, tangerine extract, lime extract, grapefruit extract and any combination thereof.

5. The method of claim 1, wherein the fine powder comprises crushed pits and seeds of fruit selected from the group consisting of apricots, olives, peaches, plums, citrus fruits and any combination thereof.

6. The cosmetic candle of claim 1, wherein the fine powder comprises a material selected from the group consisting of solid salt, solid sugar, solid salts from the Dead Sea and any combination thereof.

7. The method of claim 1, wherein the wax in the matrix is selected from the group consisting of palm kernel wax, beeswax, propolis wax, carnauba wax, and paraffin waxes.
